(19) United States
(12) Patent Application Publication
(10) Pub. No.: US 2007/0122892 A1
(43) Pub. Date: May 31, 2007

(54) Process for producing succinic acid from sucrose

(76) Inventors: Christian Andersson, Lulea (SE); Ulrika Rova, Boden (SE); Kris Arvid Berglund, Lulea (SE)

Correspondence Address: Ian C. McLeod;McLeod & Moyne, P.C., 2190 Commons Parkway, Okemos, MI, 48864, US

(21) Appl. No.: 11/290,066

(22) Filed: Nov. 30, 2005

(51) Int. Cl. C12P 7/46; C07H 1/00

(52) U.S. Cl. 435/145; 536/124

(57) ABSTRACT

A process for hydrolyzing sucrose to glucose and fructose using succinic acid is described. The hydrolysate can be used to produce purified glucose and/or fructose or can be used as a carbon source for fermentations to produce various chemicals including succinic acid.

### CROSS-REFERENCE TO RELATED APPLICATIONS

Not Applicable

### STATEMENT REGARDING FEDERALLY SPONSORED RESEARCH OR DEVELOPMENT

Not Applicable

### STATEMENT REGARDING GOVERNMENT RIGHTS

Not Applicable

### BACKGROUND OF THE INVENTION

(1) Field of the Invention

The present invention relates to a process for producing glucose and fructose from sucrose by hydrolysis using succinic acid. The hydrolysate can be used to produce the glucose/fructose or can be used as a carbon source for fermentations.

(2) Description of the Related Art

The use of inorganic acids, such as hydrochloric acid, to hydrolyze sucrose to glucose and fructose is known to those skilled in the art. An illustrative process is described in published U.S. application 2004 0231662 to De Mendonca Ferreira et al. The prior art has generally focussed on enzymatic hydrolysis of sucrose to produce glucose and/or fructose as evidenced by U.S. Pat. Nos. 5,998,177 and 6,660,502 to Catani et al.

Succinic acid is an organic acid with two carboxylic groups. It is produced today mainly petrochemically from butane through maleic anhydride. Succinic acid is currently a low volume chemical. By production of succinic acid using biomass instead of petrochemicals as raw material, many new applications are possible. The production does not contribute to the accumulation of greenhouse gases since the feedstocks are renewable. A report from the U.S. Department of Energy designates succinic acid as a top twelve building block chemical produced from biomass (Werpy T., et al., Top Value Added Chemicals from Biomass. U.S. Department of Energy: Oak Ridge (2004)). Succinic acid can be used as a commodity or specialty chemical according to the report. As a commodity chemical it can substitute chemicals based on benzene and other intermediate petrochemicals to for instance produce polyester, solvents and other acids. Food ingredients, fuel additives and environmentally benign deicers are examples of specialty chemicals from succinic acid. The feedstocks of interest for producing succinic acid contain starch, hemicellulose or cellulose and can come from agricultural residues like corn fibres, forest products or beat and cane sugar.

Fermentation can be accomplished with a number of different organisms. E. coli mutant AFP184 (described elsewhere) is used. The sugar available in sugar beet or cane is sucrose. It is a disaccharide of glucose and fructose. E. coli are not able to utilize sucrose, but can utilize glucose and fructose. Therefore, it is necessary to hydrolyze the sucrose before fermentation. Hydrolysis is normally performed with concentrated or dilute mineral acids. These acids are added to the process and require separate and costly recovery processes.

Bacteria which enable the production of succinic acid are well known. Examples are U.S. Pat. No. 6,265,190 to Yedur, U.S. Pat. No. 6,743,610 to Donnelly et al. This acid has not been used for the production of glucose and fructose.

### OBJECTS

There is a need for an improved process for the preparation of glucose and fructose from sucrose. It is therefore an object of the present invention to provide a process for hydrolysis of sucrose which uses unique acid hydrolysis step for producing glucose and sucrose. It is further an object of the present invention to provide a unique hydrolysate which can be used for fermentation processes. Further still, it is an object of the present invention to provide a process for the isolation of pure glucose and fructose. Further still, it is an object of the present invention to provide a process which provides high yields and is economical. These and other objects will become increasingly apparent by reference to the following description and the drawings.

### SUMMARY OF INVENTION

The invention provides a process which uses succinic acid to hydrolyze (invert) sucrose into fructose and glucose. The resulting sugar mixture is then used as the substrate for an E. coli or other bacterial fermentation to produce succinic acid or other organic compounds. The process is novel in that: a) it uses succinic acid for sucrose hydrolysis, which has not been previously demonstrated, and b) the resulting sugar mixture can be fermented by succinate producing organisms that are otherwise unable to produce succinic acid from sucrose. If it is desired to recover the succinic acid, it can be accomplished in the same process used for the purification of the fermentation broth to produce pure succinic acid and there would be no need for building a new recovery plant.

Thus the present invention relates to a process for producing glucose and fructose which comprises:

hydrolyzing a composition comprising sucrose in an aqueous solution with succinic acid to produce glucose and sucrose in a hydrolysate. Preferably the hydrolysis is at a temperature between about 25° C. and below a boiling point of the solution. Preferably the temperature is between about 60° and 100° C. Preferably the sucrose is at a weight ratio of between about 10 to 1 to 1 to 10 in the aqueous solution. Preferably the sucrose is at a weight ratio of between 1 to 10 and 4 to 10. Preferably the succinic acid is at a concentration between about 0.1 and 10 percent by weight in the aqueous solution. Preferably the succinic acid is at a concentration of 0.5 to 5% by weight of the aqueous solution. Preferably the glucose and fructose are produced at a yield above 95% by weight based upon a weight of glucose and fructose available in the sucrose. Preferably the hydrolysis is over a time period of up to about 24 hours. Preferably in addition the hydrolysate is fermented by a microorganism which metabolizes the glucose and fructose to produce a fermentation product. Preferably the microorganism produces succinic acid from the hydrolysate as the fermentation product. Preferably the microorganism is an Escherichia coli strain.

The present invention also relates to a fermentation process wherein a microorganism ferments a carbohydrate to produce a fermentation product, the improvement which comprises fermenting a succinic acid hydrolysate of a composition comprising sucrose to produce the fermentation product. Preferably the fermentation product is succinic acid and the microorganism is an Escherichia coli strain which with the carbohydrate produces succinic acid.

### BRIEF DESCRIPTION OF DRAWINGS

FIG. 1 is a graph showing sucrose hydrolysis at 60° C. and 1, 3, and 5% succinic acid by weight. SA is succinic acid.

FIG. 2 is a graph showing sucrose hydrolysis at 80° C. for 0.5, 1, 3, and 5% catalyst.

FIG. 3 is a graph showing sucrose hydrolysis at 100° C. for 0.5, 1, 3, and 5% catalyst.

FIG. 4 is a graph showing sucrose hydrolysis at 80° C. and 100° C. for acid concentrations of 1 and 5%.

FIG. 5 is a graph showing glucose and fructose concentrations as a function of time.

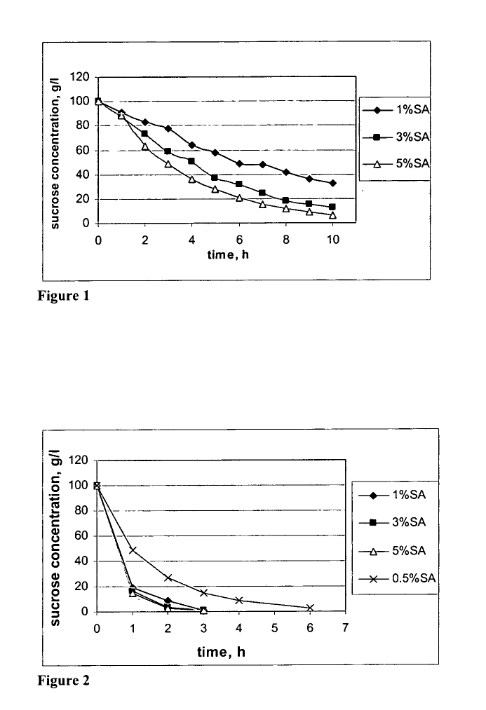

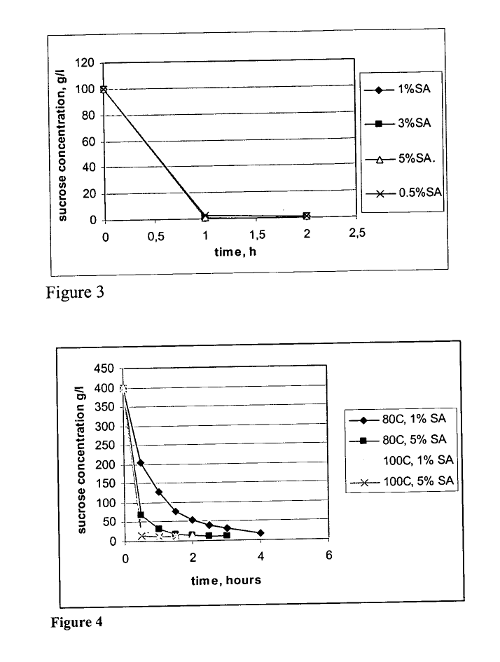

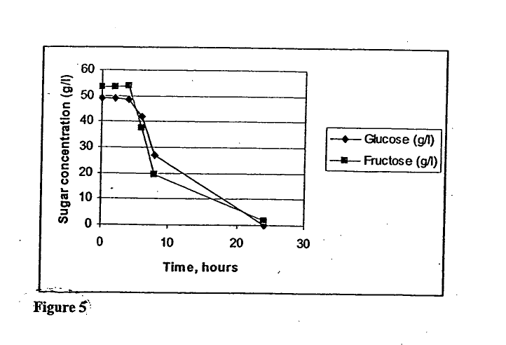

### DESCRIPTION OF PREFERRED EMBODIMENTS

The following Examples 1 to 4 show the use of succinic acid to hydrolyze sucrose to glucose and fructose in high yields.

### EXAMPLE I

Effect of Catalyst Concentration at Low Temperatures.

Sucrose and water are mixed in E-flasks in ratio liquid:solid 10:1. Temperature is kept constant at 60° C. in an oven equipped with a shake table. Succinic acid is added to the flasks to give concentrations of 1, 3, and 5% acid by weight. The data is shown in Tables 5 to 7. An increase in the catalyst loading increases the rate of hydrolysis significantly at low temperatures. Yields after ten (10) hours hydrolysis for glucose and fructose are summarized in Table 1. The yields are based on the amount of glucose or fructose formed divided by the amount of sucrose hydrolyzed. For glucose at higher acid concentrations some product degradation takes place. For fructose some degradation is evident at all acid concentrations.

TABLE 1
Yield of fermentable sugars in weight-%.

Yield (weight-%)

| | Fructose | Glucose |
|---|---|---|
| 1% SA | 88.4 | 99.3 |
| 3% SA | 86.5 | 95 |
| 5% SA | 87.2 | 94.9 |

### EXAMPLE 2

Effect of Catalyst Concentration at Elevated Temperatures

Sucrose and water were mixed in E-flasks in ratio liquid:solid 10:1. Temperature was kept constant by heating on a heating plate. Agitation was obtained from a magnetic stirrer. Temperatures investigated were 80° C. and 100° C. The catalyst loading (succinic acid added) corresponds to 0.5, 1, 3, and 5% by weight as in Example 1. Sucrose hydrolysis as a function of time for the different catalyst loadings at 80° C. and at 100° C. The data is shown in Tables 5 to 7. At higher temperatures the hydrolysis rate was greatly increased. At 80° C. acid concentrations above 1% resulted in almost complete hydrolysis after two hours. A concentration of 1% needed three hours to complete the hydrolysis and 0.5% was not finished until after six hours. At 100° C. the hydrolysis was completed in one hour for all acid concentrations. Hydrolysis products were formed in yields 95-100% based on the sucrose hydrolyzed with less degradation of fructose.

### EXAMPLE 3

Hydrolysis of Liquid:Solid Ratios 10:4

To produce a media with high enough sugar concentration for fermentation purposes higher liquid:solid ratios (higher percentages of sucrose) must be used. Using the same experimental setup as in Example 2, but with liquid:solid ratio 10:4, acid concentrations of 1%, and 5% were investigated at 80° C. and 100° C. Sucrose hydrolysis results are presented in Table 14. Yields after finished hydrolysis are shown in Table 2. The yields are expressed on a weight basis as the mass of produced monosaccharides per mass of hydrolyzed sucrose. From the yields it can be seen that the best results are achieved by low acid loadings and high temperatures. After one hour the yields were close to 100%, and heating for another hour did not generate any significant sugar degradation.

TABLE 2
Yield of fermentable sugars in weight-%.

Yield (weight-%)

| | Glucose | Fructose |
|---|---|---|
| 80° C., 1% SA | 81.8 | 86.2 |
| 80° C., 5% SA | 82.3 | 86.2 |
| 100° C., 1% SA | 95.9 | 99.4 |
| 100° C., 5% SA | 88.8 | 92.3 |

### EXAMPLE 4

Fermentation of Glucose-Fructose Mixture in Ratio 50-50

A 12 L fermenter was used. A media constituted by the substances given in Table 3 was mixed and sterilized in the fermenter. 0.5 L inoculum of a pure AFP184 (an E. coli mutant described in U.S. published application 200300/7559 to Donnelly et al), grown for 16 hours in sterile Tryptic Soy Broth, was added together with a 2 L solution with a concentration of 400 g/L of glucose and fructose in ratio 1:1. The total starting volume of the fermentation was 8 L. The fermentation was aerated for eight hours with filter sterilized air. After eight hours a high cell density was achieved and the fermentation conditions were changed to anaerobic by cutting of air and adding carbon dioxide. This then proceeds for 16 hours producing succinic acid. The final succinic acid concentration, yield per gram and mole consumed sugar during the anaerobic phase and the productivity per gram, litre and hour is shown in Table 4. The sugar concentration change during the fermentation is presented in Table 15. E. coli mutant AFP184 can ferment mixtures of glucose and fructose as produced in Examples 1 to 3.

TABLE 3
Mineral and growth factor substrates for fermentation.

| Substrate | Amount (g or L) | |
|---|---|---|
| Corn Steep | 266 | g |
| Liquor | | |
| K₂HPO₄ | 11.2 | g |
| KH₂PO₄ | 4.8 | g |
| (NH₄)₂SO₄ | 26.7 | g |
| MgSO₄ | 1.6 | g |
| Antifoam agent | 3 | ml |

TABLE 4
Final succinic acid concentration, yields, and
productivity after 16 hours of production.

| Succinic Acid | Yield | Yield | Productivity |
|---|---|---|---|
| (g/L) | (g/g) | (mole/mole) | (g/L/h) |
| 51.95 | 0.73 | 1.12 | 2.89 |

### Data—Example 1

TABLE 5
Glucose formation (g/L) at 60° C.
for acid concentrations 1, 3, and 5%.

| time | Glucose, 1% SA | Glucose, 3% SA | Glucose, 5% SA |
|---|---|---|---|
| 0 | 0 | 0 | 0 |
| 1 | 0 | 0.665254592 | 1.433727361 |
| 2 | 2.647401928 | 8.319265668 | 12.49919376 |
| 3 | 8.943332524 | 15.28128695 | 20.67329705 |
| 4 | 11.81570429 | 22.30937982 | 27.23962543 |
| 5 | 19.77152651 | 26.05398002 | 35.49323757 |
| 6 | 17.31541166 | 33.45268288 | 38.71977609 |
| 7 | 27.57577628 | 35.48615065 | 40.78236791 |
| 8 | 28.83985555 | 34.19917824 | 42.36255161 |
| 9 | 30.72163142 | 39.90952235 | 43.57539008 |
| 10 | 33.34785223 | 41.47563175 | 44.24743298 |

TABLE 6
Fructose formation (g/L) at 60° C.
for acid concentrations 1, 3, and 5%.

| time | Fructose, 1% SA | Fructose, 3% SA | Fructose, 5% SA |
|---|---|---|---|
| 0 | 0 | 0 | 0 |
| 1 | 0.67972 | 2.33298 | 3.65876 |
| 2 | 3.837 | 9.80845 | 12.9257 |
| 3 | 8.35472 | 16.32199 | 20.89805 |
| 4 | 12.95957 | 22.18015 | 26.83508 |
| 5 | 16.86656 | 26.67031 | 32.28647 |
| 6 | 20.14169 | 30.14726 | 34.66879 |
| 7 | 24.08336 | 32.67965 | 37.59368 |
| 8 | 25.38129 | 34.36781 | 39.20524 |
| 9 | 27.23786 | 36.60941 | 39.96632 |
| 10 | 29.69138 | 37.77904 | 40.66125 |

TABLE 7
Sucrose degradation (g/L) at 60° C.
for acid concentrations 1, 3, and 5%.

| time | Sucrose, 1% SA | Sucrose, 3% SA | Sucrose, 5% SA |
|---|---|---|---|
| 0 | 100 | 100 | 100 |
| 1 | 91.47598829 | 87.58720289 | 88.40802667 |
| 2 | 82.73131168 | 73.10737983 | 63.0472783 |
| 3 | 77.27112921 | 58.94906893 | 48.72138111 |
| 4 | 64.26281948 | 50.48299621 | 35.88196376 |
| 5 | 57.96055181 | 36.76323165 | 27.96802436 |
| 6 | 48.86978287 | 31.59578454 | 20.82431382 |
| 7 | 47.39983256 | 24.79683883 | 15.29118304 |
| 8 | 41.42066148 | 18.43986528 | 11.84848076 |
| 9 | 35.90348212 | 15.58531013 | 8.835096554 |
| 10 | 32.80338213 | 12.68737568 | 6.722507349 |

### Data—Example 2

TABLE 8
Sucrose degradation at 80° C. for
acid concentrations 0.5, 1, 3, and 5%.

| time | 0.5% SA | 1% SA | 3% SA | 5% SA |
|---|---|---|---|---|
| 0 | 100 | 100 | 100 | 100 |
| 1 | 48.53388 | 19.12333554 | 16.81258471 | 14.88987095 |
| 2 | 27.26248 | 8.703267151 | 3.326631595 | 2.8984055 |
| 3 | 14.79558 | 0.881246481 | 0.881246481 | 0.881246481 |
| 4 | 8.854452 | | | |
| 6 | 2.418129 | | | |

TABLE 9
Glucose formation at 80° C. for acid
concentrations 0.5, 1, 3, and 5%.

| time | 0.5% SA | 1% SA | 3% SA | 5% SA |
|---|---|---|---|---|
| 0 | 0 | 0 | 0 | 0 |
| 1 | 18.7314 | 39.52014 | 41.16771 | 35.40328 |
| 2 | 33.2953 | 43.74562 | 50.29046 | 44.26493 |
| 3 | 40.46413 | 45.00098 | 48.96617 | 45.14757 |
| 4 | 48.98223 | | | |
| 6 | 48.27418 | | | |

TABLE 10
Fructose formation at 80° C. for
acid concentrations 0.5, 1, 3, and 5%.

| time | 0.5% SA | 1% SA | 3% SA | 5% SA |
|---|---|---|---|---|
| 0 | 0 | 0 | 0 | 0 |
| 1 | 17.85198 | 37.612 | 42.35413 | 38.71786 |
| 2 | 34.52418 | 47.15255 | 48.16839 | 47.37839 |
| 3 | 42.22377 | 47.65608 | 49.9901 | 48.4048 |
| 4 | 51.25258 | | | |
| 6 | 51.51636 | | | |

TABLE 11
Sucrose degradation at 100° C. for
acid concentrations 0.5, 1, 3, and 5%.

| time | 0.5% SA | 1% SA | 3% SA | 5% SA |
|---|---|---|---|---|
| 0 | 100 | 100 | 100 | 100 |
| 1 | 2.44425 | 0.88125 | 0.88125 | 0.88125 |
| 2 | 0.881246 | 0.88125 | | 0.88125 |

TABLE 12
Glucose formation at 100° C. for
acid concentrations 0.5, 1, 3, and 5%.

| time | 0.5% SA | 1% SA | 3% SA | 5% SA |
|---|---|---|---|---|
| 0 | 0 | 0 | 0 | 0 |
| 1 | 52.84148 | 52.02847 | 53.60736 | 46.82761 |
| 2 | 56.9705 | 49.00942 | | 47.37418 |

TABLE 13
Fructose formation at 100° C. for
acid concentrations 0.5, 1, 3, and 5%.

| time | 0.5% SA | 1% SA | 3% SA | 5% SA |
|---|---|---|---|---|
| 0 | 0 | 0 | 0 | 0 |
| 1 | 55.07813 | 54.49409 | 55.61529 | 49.07266 |
| 2 | 59.56918 | 51.47787 | | 50.42975 |

### Data—Example 3

TABLE 14
Sucrose degradation, glucose and fructose formation at 80° C. and 100° C.,
1% and 5% succinic acid and liquid solid ratio 10:4 in g/L.

| | Time | | | | | | | |
|---|---|---|---|---|---|---|---|---|
| | 0 | 0.5 | 1 | 1.5 | 2 | 2.5 | 3 | 4 |
| Sucrose 80 C., 1% SA | 400 | 205.58 | 127.32 | 77.34 | 54.09 | 38.98 | 30.49 | 18.14 |
| Sucrose 80 C., 5% SA | 400 | 67.60 | 32.44 | 16.95 | 13.81 | 12.59 | 12.59 | |
| Sucrose 100 C., 1% SA | 400 | 28.41 | 13.09 | 12.59 | 12.59 | | | |
| Sucrose 100 C., 5% SA | 400 | 14.52 | 12.80 | 12.59 | | | | |
| Glucose 80 C., 1% SA | 0 | 57.60 | 104.19 | 121.73 | 137.28 | 147.30 | 169.42 | 156.22 |
| Glucose 80 C., 5% SA | 0 | 145.39 | 152.11 | 171.20 | 177.72 | 175.63 | 159.33 | |
| Glucose 100 C., 1% SA | 0 | 145.47 | 179.85 | 187.19 | 185.72 | | | |
| Glucose 100 C., 5% SA | 0 | 174.43 | 177.97 | 172.02 | | | | |
| Fructose 80 C., 1% SA | 0 | 61.36 | 101.61 | 124.35 | 141.85 | 154.89 | 177.06 | 164.61 |
| Fructose 80 C., 5% SA | 0 | 151.39 | 159.09 | 178.22 | 185.40 | 184.48 | 166.88 | |
| Fructose 100 C., 1% SA | 0 | 152.44 | 187.22 | 195.90 | 192.62 | | | |
| Fructose 100 C., 5% SA | 0 | 181.21 | 186.07 | 178.86 | | | | |

| Time | Glucose (g/L) | Fructose (g/L) |
|---|---|---|
| 0 | 48.96 | 53.18 |
| 2 | 48.9 | 53.19 |
| 4 | 48.57 | 53.65 |
| 6 | 41.92 | 37.65 |
| 8 | 26.83 | 19.23 |
| 24 | 0 | 1.68 |

### Data—Example 4

Various sources of sucrose can be used. In some instances the source can contain other materials as in the case of molasses.

It is intended that the foregoing description be only illustrative of the present invention and that the present invention be limited only by the hereinafter appended claims.

I claim:

1. A process for producing glucose and fructose which comprises:
hydrolyzing a composition comprising sucrose in an aqueous solution with succinic acid to produce glucose and sucrose in a hydrolysate.

2. The process of claim 1 wherein the hydrolysis is at a temperature between about 25° C. and below a boiling point of the solution.

3. The process of claim 1 wherein the temperature is between about 60° and 100° C.

4. The process of claims 1 or 2 wherein the sucrose is at a weight ratio of between about 10 to 1 to 1 to 10 in the aqueous solution.

5. The process of claims 1 or 2 wherein the sucrose is at a weight ratio of between 1 to 10 and 4 to 10.

6. The process of claims 1 or 2 wherein the succinic acid is at a concentration between about 0.1 and 10 percent by weight in the aqueous solution.

7. The process of claims 1 or 2 wherein the succinic acid is at a concentration of 0.5 to 5% by weight of the aqueous solution.

8. The process of claims 1 or 2 wherein the glucose and fructose are produced at a yield above 95% by weight based upon a weight of glucose and fructose available in the sucrose.

9. The process of claims 1 or 2 wherein the hydrolysis is over a time period of up to about 24 hours.

10. The process of claim 1 wherein in addition the hydrolysate is fermented by a microorganism which metabolizes the glucose and fructose to produce a fermentation product.

11. The process of claim 10 wherein the microorganism produces succinic acid from the hydrolysate as the fermentation product.

12. The process of claim 11 wherein the microorganism is an Escherichia coli strain.

13. In a fermentation process wherein a microorganism ferments a carbohydrate to produce a fermentation product, the improvement which comprises fermenting a succinic acid hydrolysate of a composition comprising sucrose to produce the fermentation product.

14. The process of claim 13 wherein the fermentation product is succinic acid and the microorganism is an Escherichia coli strain which with the carbohydrate produces succinic acid.
